(19) United States
(12) Patent Application Publication
(10) Pub. No.: US 2019/0216554 A1
(43) Pub. Date: Jul. 18, 2019

(54) STERILE ADAPTER ASSEMBLY FOR A ROBOTIC SURGICAL SYSTEM

(71) Applicant: SS Innovations China Co. Ltd., Hangzhou (CN)

(72) Inventor: Salman Kapadia, Barapatthar (IN)

(73) Assignee: SS Innovations China Co. Ltd., Hangzhou (CN)

(21) Appl. No.: 16/245,844

(22) Filed: Jan. 11, 2019

(30) Foreign Application Priority Data
Jan. 12, 2018 (IN) 201811001409
Sep. 28, 2018 (IN) 201812036194

(51) Int. Cl. A61B 34/30; A61B 46/10

(52) U.S. Cl. CPC A61B 34/30; A61B 34/71; A61B 46/10

(57) ABSTRACT

A sterile adapter assembly (303) is disclosed herein. The sterile adapter assembly (303) comprises of a housing (401) and a floating plate (423) positioned within the housing (401). The sterile adapter assembly further comprises of at least one rotatable body (505a) having a circumferential surface affixed on the floating plate (423). The at least one rotatable body (505a) having at least one opening (601a) capable of receiving at least one pin of a driving element (413) of an actuator assembly (305). The sterile adapter assembly further comprises of a compression mechanism (700) positioned inside the at least one rotatable body (505a) to align the at least one pin of the driving element (413) of the actuator assembly (305) with the at least one opening (601a) of the at least one rotatable body (505a).

### FIELD OF THE INVENTION

The present invention generally relates to a robotic surgical system for minimally invasive surgery. More particularly, the invention relates to an improved sterile adapter assembly in the robotic surgical system.

### BACKGROUND OF THE INVENTION

This section is intended to introduce the reader to various aspects of art that may be related to various aspects of the present disclosure, which are described below. This disclosure is believed to be helpful in providing the reader with background information to facilitate a better understanding of the various aspects of the present disclosure. Accordingly, it should be understood that these statements are to be read in this light, and not just as admissions of prior art.

Robotically assisted surgical systems have been adopted worldwide to replace conventional surgical procedures to reduce amount of extraneous tissue(s) that may be damaged during surgical or diagnostic procedures, thereby reducing patient recovery time, patient discomfort, prolonged hospital tenure, and particularly deleterious side effects. In robotically assisted surgeries, the surgeon typically operates a master controller at a surgeon console to seamlessly capture and transfer complex actions performed by the surgeon giving the perception that the surgeon is directly articulating surgical tools to perform the surgery. The surgeon operating on the surgeon console may be located at a distance from a surgical site or may be located within an operating theatre where the patient is being operated.

The robotically assisted surgeries have revolutionized the medical field and one of the fastest growing sectors in medical device industry. However, the major challenge in robotically assisted surgeries is to ensure the safety and precision during the surgery. One of the key areas of robotically assisted surgeries is the development of surgical robots for minimally invasive surgery. Over the last couple of decades, surgical robots have evolved exponentially and has been a major area of innovation in the medical device industry.

The robotically assisted surgical systems comprises of multiple robotic arms aiding in conducting robotic surgeries. The robotically assisted surgical system utilizes a sterile barrier to separate the non-sterile section of the robotic arm from a mandatory sterile surgical instrument attached to the robotic arm at an operating end. The sterile barrier may include a sterile plastic drape that envelops the robotic arm and a sterile adapter that operably engages with the sterile surgical instrument in a sterile field. The sterile barrier also may include a flexing drape interface to retain a drape section therebetween such that the torque and other force feedbacks is received as an input from both the sterile surgical instrument as well as the robotic arm. The sterile barrier is maintained between the sterile surgical instrument and the non-sterile robotic system. The sterile adapter detachably engages with an actuator assembly which drives and controls the sterile surgical instrument in a sterile field.

Performing surgery with surgical instruments in robotic surgeries creates new challenges. One challenge is the need to maintain the region adjacent the patient in a sterile condition. However, electrical components in the actuator assembly such as motors, sensors, encoders, and electrical connections that are necessary to control and move the sterile surgical instruments typically cannot be sterilized using conventional methods, e.g., steam, heat and pressure or chemicals, because they would be damaged or destroyed in the respective sterilization process.

Thus, it's imperative that an easier and more effective way to engage and disengage the sterile surgical instrument from the sterile barrier and thereon from the actuator assembly while preventing contamination of the actuator assembly and allowing quick and reliable attachment of a succession of sterile surgical instruments from the sterile barrier that maintains a sterile area around the surgical instrument.

Another challenge in the robotically assisted surgical system is easy engagement and disengagement of the sterile adapter from the actuator assembly such that the sterile barrier is not breached. However, the interlocking between the sterile adapter and the actuator assembly involves complex assembly thereby making the assembly costly, cumbersome, and time consuming.

In the light of aforementioned challenges, there is a need for a robotic surgical system with improved sterile adapter assembly that allows easy attachability/detachability of the sterile adapter assembly and the actuator assembly during performance of the robotic surgery without breaking the sterile barrier.

### SUMMARY OF THE INVENTION

A sterile adapter assembly (303) is disclosed herein. The sterile adapter assembly (303) comprises of a housing (401) and a floating plate (423) positioned within the housing (401). The sterile adapter assembly further comprises of at least one rotatable body (505a) having a circumferential surface affixed on the floating plate (423). The at least one rotatable body (505a) having at least one opening (601a) capable of receiving at least one pin of a driving element (413) of an actuator assembly (305). The sterile adapter assembly further comprises of a compression mechanism (700) positioned inside the at least one rotatable body (505a) to align the at least one pin of the driving element (413) of the actuator assembly (305) with the at least one opening (601a) of the at least one rotatable body (505a).

### BRIEF DESCRIPTION OF THE DRAWINGS

To further clarify advantages and features of the present invention, a more particular description of the invention will be rendered by reference to specific embodiments thereof, which is illustrated in the appended drawings. It is appreciated that these drawings depict only typical embodiments of the invention and are therefore not to be considered limiting of its scope. The invention will be described and explained with additional specificity and detail with the accompanying drawings in which:

FIG. 1(a) illustrates a schematic diagram of multiple robotic arms of a robotic surgical system in accordance with an embodiment of the invention;

FIG. 1(b) illustrates a schematic diagram of a surgeon console of the robotic surgical system in accordance with an embodiment of the invention;

FIG. 1(c) illustrates a schematic diagram of a vision cart of the robotic surgical system in accordance with an embodiment of the invention;

FIG. 2 illustrates a perspective view of a tool interface assembly mounted on a robotic arm in accordance with an embodiment of the invention;

FIG. 3(a) illustrates a perspective view of the tool interface assembly in accordance with an embodiment of the invention;

FIG. 3(b) illustrates an exploded view of the tool interface assembly in accordance with an embodiment of the invention;

FIG. 4(a) illustrates a front view of an actuator assembly and a sterile adapter assembly in the detached position in accordance with an embodiment of the invention;

FIG. 4(b) illustrates a rear view of the actuator assembly and the sterile adapter assembly in the detached position in accordance with an embodiment of the invention;

FIG. 4(c) illustrates a view of the actuator assembly and the sterile adapter assembly in the locked position in accordance with an embodiment of the invention;

FIG. 5(a) illustrates a top view of the sterile adapter assembly in accordance with an embodiment of the invention;

FIG. 5(b) illustrates a bottom view of the sterile adapter assembly in accordance with an embodiment of the invention;

FIG. 6(a) illustrates a front view of a floating plate of the sterile adapter assembly in accordance with an embodiment of the invention;

FIG. 6(b) illustrates a rear view of the floating plate of the sterile adapter assembly in accordance with an embodiment of the invention;

FIG. 7 illustrates a perspective view of the floating plate without a rotatable body in accordance with an embodiment of the invention;

FIG. 8(a) illustrates the rotatable body with a spring in accordance with an embodiment of the invention; and

FIG. 8(b) illustrates isolated view of the spring as shown in the FIG. 8(a) in accordance with an embodiment of the invention.

### DETAILED DESCRIPTION OF THE INVENTION

For the purpose of promoting an understanding of the principles of the invention, reference will now be made to the embodiment illustrated in the drawings and specific language will be used to describe the same. It will nevertheless be understood that no limitation of the scope of the invention is thereby intended, such alterations and further modifications in the illustrated system, and such further applications of the principles of the invention as illustrated therein being contemplated as would normally occur to one skilled in the art to which the invention relates.

It will be understood by those skilled in the art that the foregoing general description and the following detailed description are exemplary and explanatory of the invention and are not intended to be restrictive thereof. Throughout the patent specification, a convention employed is that in the appended drawings, like numerals denote like components.

Reference throughout this specification to “an embodiment”, “another embodiment”, “an implementation”, “another implementation” or similar language means that a particular feature, structure, or characteristic described in connection with the embodiment is included in at least one embodiment of the present invention. Thus, appearances of the phrase “in an embodiment”, “in another embodiment”, “in one implementation”, “in another implementation”, and similar language throughout this specification may, but do not necessarily, all refer to the same embodiment.

The terms “comprises”, “comprising”, or any other variations thereof, are intended to cover a non-exclusive inclusion, such that a process or method that comprises a list of steps does not include only those steps but may include other steps not expressly listed or inherent to such process or method. Similarly, one or more devices or sub-systems or elements or structures proceeded by “comprises . . . a” does not, without more constraints, preclude the existence of other devices or other sub-systems or other elements or other structures or additional devices or additional sub-systems or additional elements or additional structures.

Unless otherwise defined, all technical and scientific terms used herein have the same meaning as commonly understood by one of ordinary skill in the art to which this invention belongs. The apparatus, system, and examples provided herein are illustrative only and not intended to be limiting.

The terms “a” and “an” herein do not denote a limitation of quantity, but rather denote the presence of at least one of the referenced item. Further, the term sterile barrier and sterile adapter denotes the same meaning and may be used interchangeably throughout the description.

Embodiments of the invention will be described below in detail with reference to the accompanying drawings.

The invention relates to a robotic surgical system for minimally invasive surgery. The robotic surgical system will generally involve the use of multiple robotic arms. One or more of the robotic arms will often support a surgical tool which may be articulated (such as jaws, scissors, graspers, needle holders, micro dissectors, staple appliers, tackers, suction/irrigation tools, clip appliers, or the like) or non-articulated (such as cutting blades, cautery probes, irrigators, catheters, suction orifices, or the like). One or more of the robotic arms will often be used to support one or more surgical image capture devices such as an endoscope (which may be any of the variety of structures such as a laparoscope, an arthroscope, a hysteroscope, or the like), or optionally, some other imaging modality (such as ultrasound, fluoroscopy, magnetic resonance imaging, or the like).

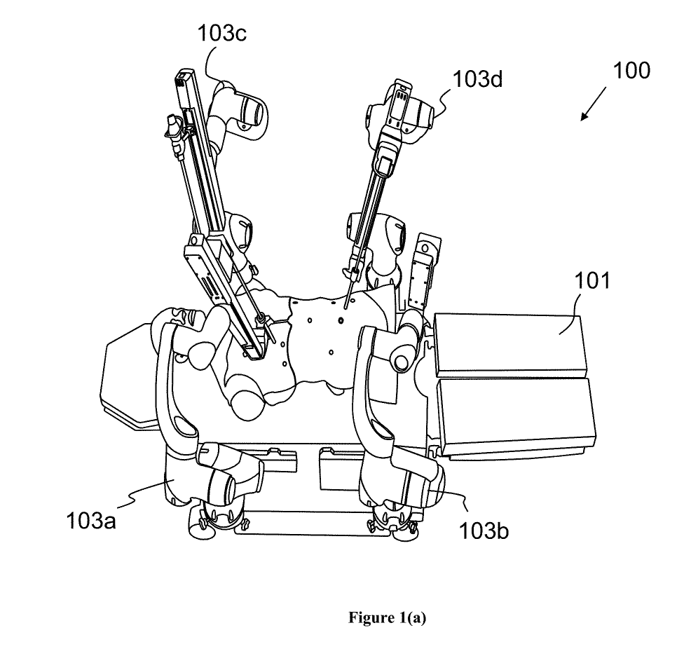

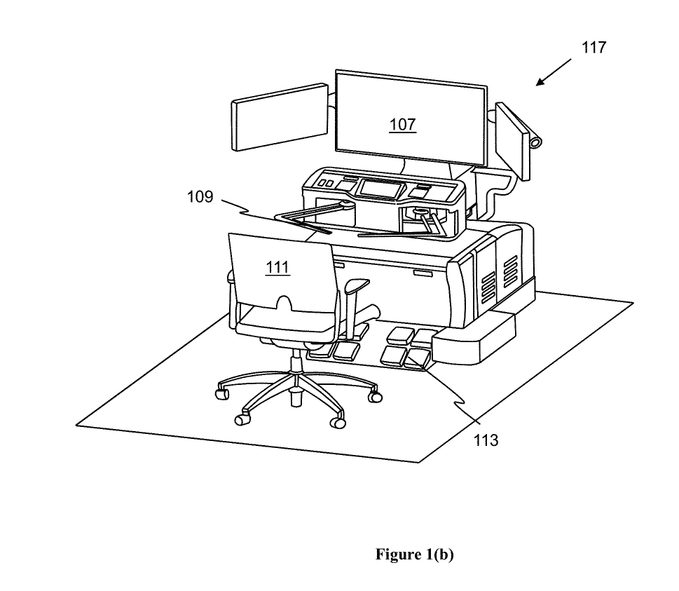

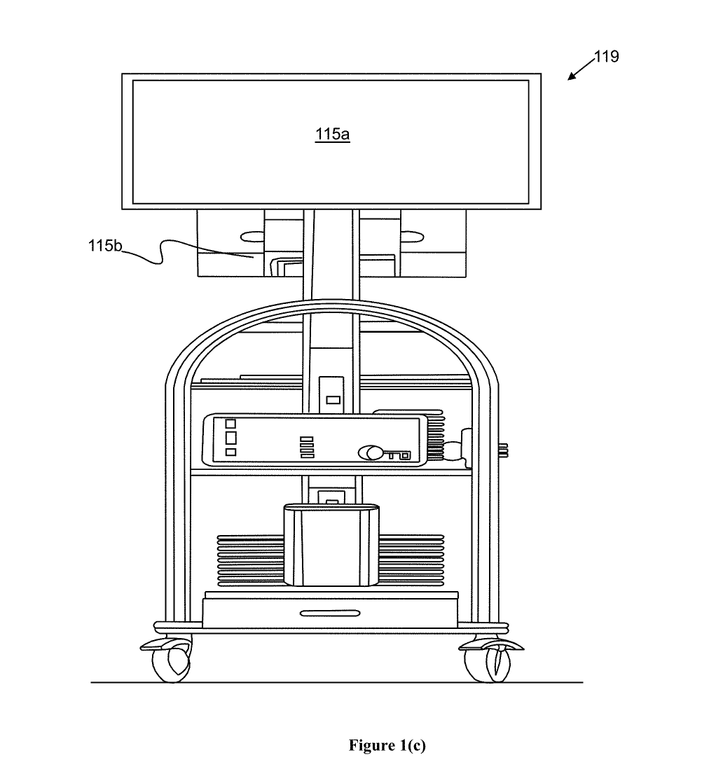

FIG. 1(a) illustrates a schematic diagram of multiple robotic arms of a robotic surgical system in accordance with an embodiment of the invention. Specifically, FIG. 1 illustrates the robotic surgical system (100) having four robotic arms (103a), (103b), (103c), (103d) mounted around a patient cart (101). The four-robotic arms (103a), (103b), (103c), (103d) as depicted in FIG. 1 is for illustration purpose and the number of robotics arms may vary depending upon the type of surgery or the robotic surgical system. The four robotic arms (103a), (103b), (103c), (103d) are mounted along the patient cart (101) and may be arranged in different manner but not limited to the robotic arms (103a), (103b), (103c), (103d) mounted on the patient cart 101 or the robotic arms (103a), (103b), (103c), (103d) separately mounted on a movable means or the robotic arms (103a), (103b), (103c), (103d) mechanically and/or operationally connected with each other or the robotic arms (103a), (103b), (103c), (103d) connected to a central body such that the robotic arms (103a), (103b), (103c), (103d) branch out of the central body (now shown).

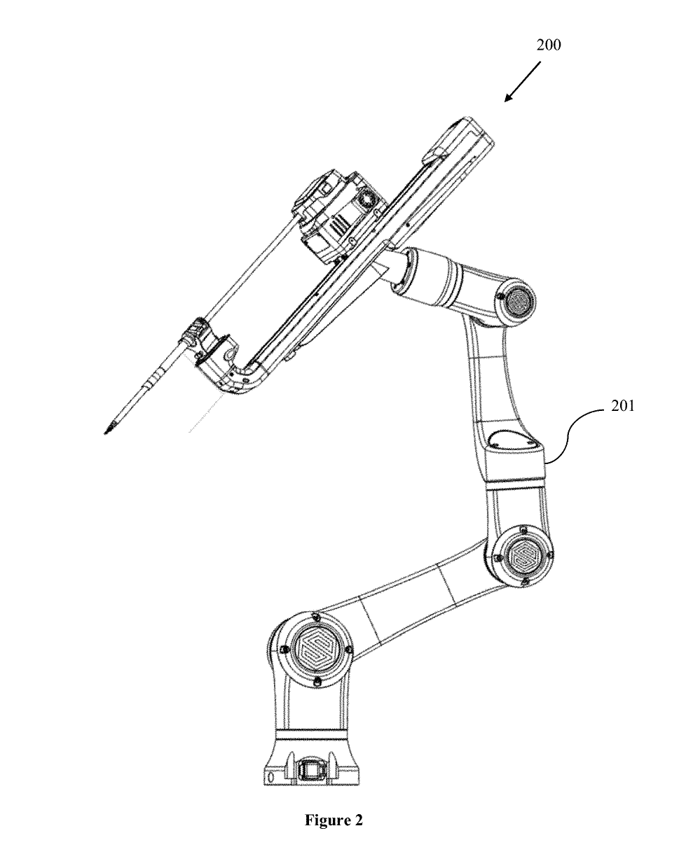

FIG. 1(b) illustrates a schematic diagram of a surgeon console of the robotic surgical system in accordance with an embodiment of the invention. The surgeon console (117) aids the surgeon to remotely operate the patient lying on the patient cart (101) by controlling the robotic arms (103a), (103b), (103c), (103d) inside the body of the patient. The surgeon console (117) is configured to control the movement of surgical instruments (as shown in FIG. 2) while the instruments are inside the patient. The surgeon console (117) may comprise of at least an adjustable viewing means (107) but not limited to 2D/3D monitors, wearable viewing means (not shown) and in combination thereof. The surgeon console (117) may be equipped with multiple displays which would not only show 3D high definition (HD) endoscopic view of a surgical site at the patient cart (101) but may also shows additional information from various medical equipment's which surgeon may use during the robotic surgery. Further, the viewing means (107) may provide various modes of the robotic surgical system (100) but not limited to identification and number of robotic arms attached, current tool type attached, current tool tip position, collision information along with medical data like ECG, ultrasound display, fluoroscopic images, CT, MRI information. The surgeon console (117) may further comprise of mechanism for controlling the robotics arms but not limited to one or more hand controllers (109), one or more foot controllers (113), a clutch mechanism (not shown), and in combination thereof. The hand controllers (109) at the surgeon console (117) are required to seamlessly capture and transfer complex actions performed by surgeon giving the perception that the surgeon is directly articulating the surgical tools. The different controllers may require for different purpose during the surgery. In some embodiments, the hand controllers (109) may be one or more manually-operated input devices, such as a joystick, exoskeletal glove, a powered and gravity-compensated manipulator, or the like. These hand controllers (109) control teleoperated motors which, in turn, control the movement of the surgical instruments attached to the robotic arms. The surgeon may sit on a resting apparatus such as a chair (111), as depicted in FIG. 1(b), while controlling the surgeon console (117). The chair (111) may be adjustable with means in height, elbow rest and the like according to the ease of the surgeon and also various control means may be provided on the chair (111). Further, the surgeon console (117) may be at a single location inside an operation theatre, or may be distributed at any other location in the hospital provided connectivity to the robotics arms is maintained.

FIG. 1(c) illustrates a schematic diagram of a vision cart of the robotic surgical system in accordance with an embodiment of the invention. The vision cart (119) is configured to display the 2D and/or 3D view of the operation captured by an endoscope. The vision cart (119) may be adjusted at various angles and heights depending upon the ease of view. The vison cart (119) may have various functionality but not limited to providing touch screen display, preview/recording/playback provisions, various inputs/outputs means, 2D to 3D converters and the like. The vision cart (119) may include a vision system portion (not shown) that enables a spectator or other non-operating surgeons to view a surgical site from outside the patient's body. One of the robotics arms typically engage a surgical instrument that has a video-image-capture function (i.e., a camera instrument) for displaying the captured images on the vision cart (119). In some robotic surgical system configurations, the camera instrument includes optics that transfer the images from the distal end of the camera instrument to one or more imaging sensors (e.g., CCD or CMOS sensors) outside of the patient's body. Alternatively, the imaging sensor(s) may be positioned at the distal end of the camera instrument, and the signals produced by the sensor(s) may be transmitted along a lead or wirelessly for processing and display on the vision cart (119).

FIG. 2 illustrates a perspective view of a tool interface assembly mounted on a robotic arm in accordance with an embodiment of the invention. The tool interface assembly (200) is mounted on the robotic arm (201) of the robotic surgical system (100). The tool interface assembly (200) is the main component for performing the robotic surgery on a patient. The robotic arm (201) as shown in FIG. 2 is shown for the illustration purpose only and other robotic arms with different configurations, degree of freedom (DOF) and shapes may be used.

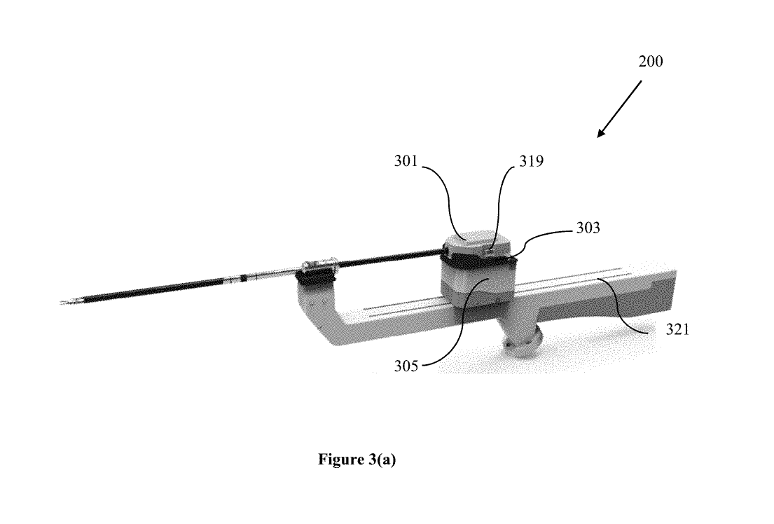

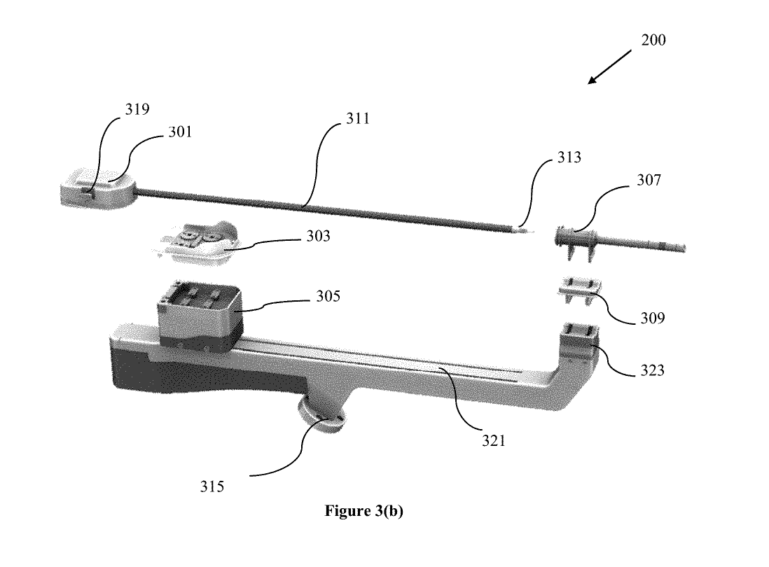

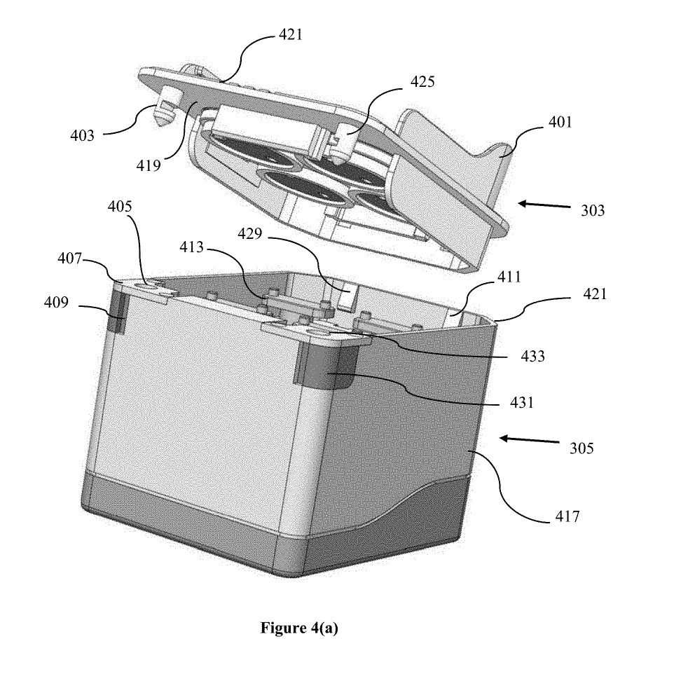

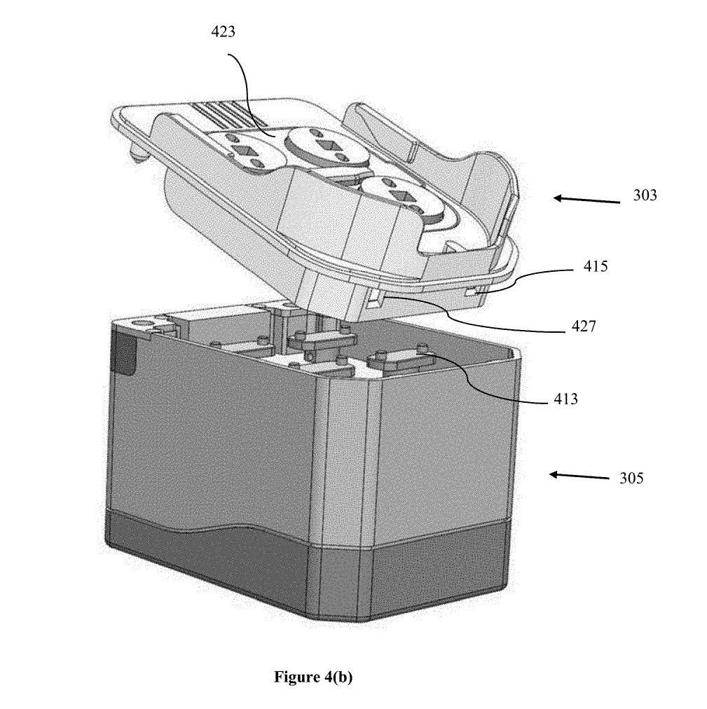

FIG. 3(a) illustrates a perspective view of the tool interface assembly and FIG. 3(b) illustrates an exploded view of the tool interface assembly in accordance with an embodiment of the invention. The tool interface assembly (200), as depicted by any of the FIG. 3(a) or FIG. 3(b), comprises of an ATI (arm and tool interface) connector (315) (shown in FIG. 3(b)) which facilitates the tool interface (200) to operationally connect with the robotic arm (shown in FIG. 2). Further, the tool interface (200) further comprises of an actuator assembly (305) mounted on a guiding mechanism and capable of linearly moving along the guiding mechanism. The guiding mechanism depicted in FIGS. 3(a) and 3(b) is a guide rail (321). The movement of the actuator assembly (305) along the guide rail (321) is controlled by the surgeon with the help of controllers on the surgeon console (117) as shown in FIG. 1(b). A sterile adapter assembly (303) is releasably mounted on the actuator assembly (305) to separate a non-sterile part of the robotic arm from a sterile surgical tool assembly (301). A locking mechanism (as shown in FIG. 4) is provided to releasably lock and unlock the sterile adapter assembly (303) with the actuator assembly (305). The sterile adapter assembly (303) detachably engages from the actuator assembly (305) which drives and controls the sterile surgical instrument in a sterile field. In another embodiment, the surgical tool assembly (301) also may be releasably lock/unlock or engages/disengages with the sterile adapter assembly (303) by means of a push button (319).

Referring now to FIG. 3(b), the surgical tool assembly (301) includes a shaft (311) and end effectors (313). The end effector (313) may comprises of a surgical instrument or may be configured to attach a surgical instrument. The end effector (313) may be a surgical instrument associated with one or more surgical tasks, such as a forcep, a needle driver, a shears, a bipolar cauterizer, a tissue stabilizer or retractor, a clip applier, an anastomosis device, an imaging device (e.g., an endoscope or ultrasound probe), and the like. Some surgical instruments further provide an articulated support (sometimes referred to as a “wrist”) for the surgical tool assembly (301) such that the position and orientation of the surgical tool assembly (301) may be manipulated with one or more mechanical degrees of freedom in relation to the instrument's shaft (311). Further, the end effectors (313) include a functional mechanical degree of freedom, such as jaws that open or close, or a knife that translates along a path. The surgical tool assembly (301) may also contain stored (e.g., on a semiconductor memory inside the instrument) information that may be permanent or may be updatable by the robotic surgical system (100).

A cannula gripper (309) is provided on the tool interface assembly (200) and is configured to grip a cannula (307) which receives the shaft (311) through an opening (not shown). The cannula (307) comprises of a hollow body which comprises of grooves (not shown) in an internal surface (not shown). The grooves provide a locking mechanism that fixes the cannula (307) to the shaft (311) at desired angle and precludes shifting, twisting or any axial movement of the shaft (311) once received by the cannula (307). The cannula gripper (309) is detachably attached to one end of the tool interface assembly (200) and comprises of flap like body which receives the cannula (307). Alternatively, the cannula gripper (309) may have a circular body for receiving the cannula (307) and comprises of grooves to grip the cannula (307) at a stationary position.

The cannula gripper (309) may be affixed to the body of the tool interface assembly (200) and may be configured to grip or secure the cannula (307) such that cannula (307) is stable while performing surgical operations. The cannula gripper (309) may be affixed to a mount (323) of the tool interface assembly (200) by way of receiving the cannula gripper (309) within a set of grooves of the mount (323).

FIG. 4(a) illustrates a front view of an actuator assembly and a sterile adapter assembly in detached position in accordance with an embodiment of the invention. The sterile adapter assembly (303) is configured to be capable of being releasably mounted on the actuator assembly (305) by means of various locking mechanisms, but not limited to snap fit, push button locking mechanism and the like.

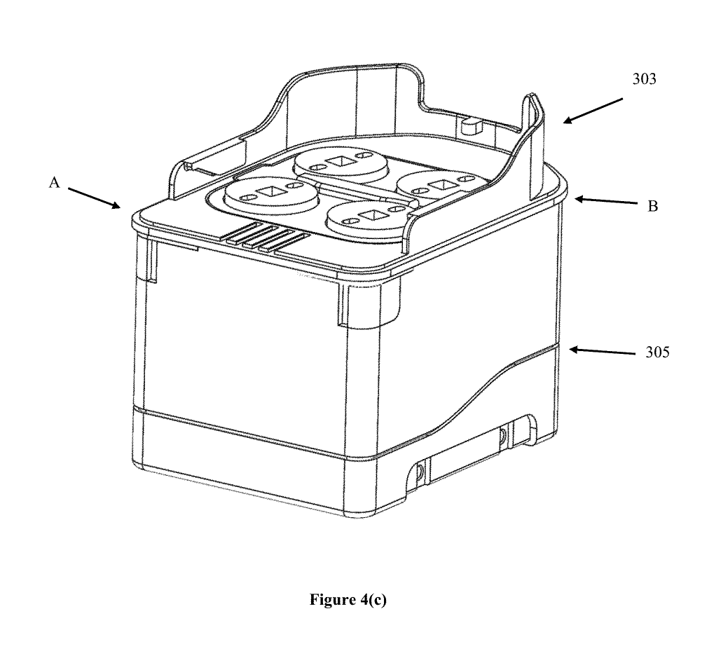

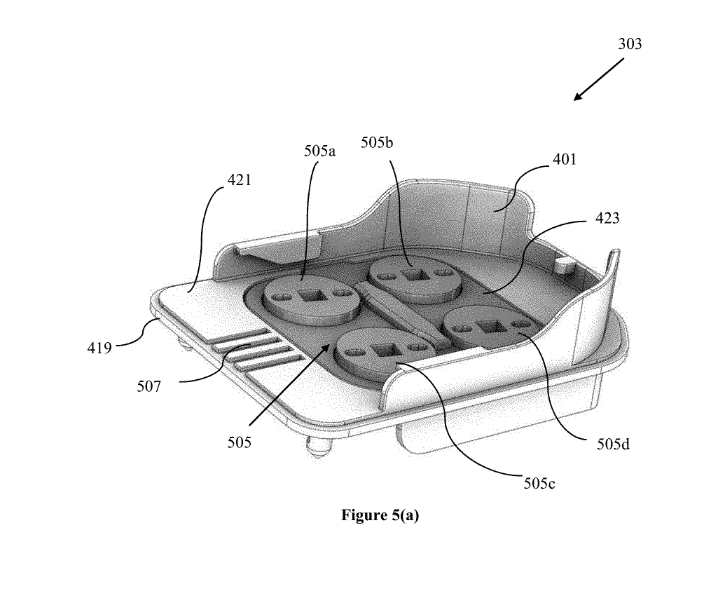

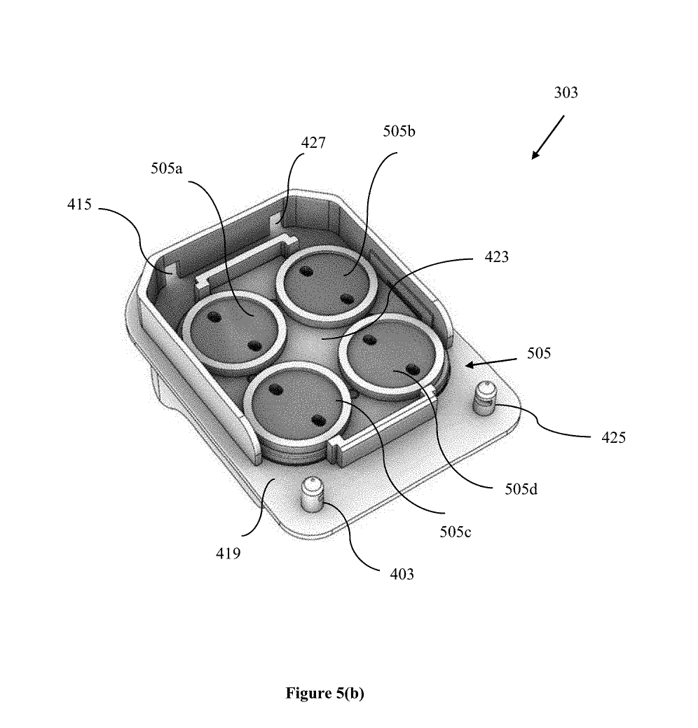

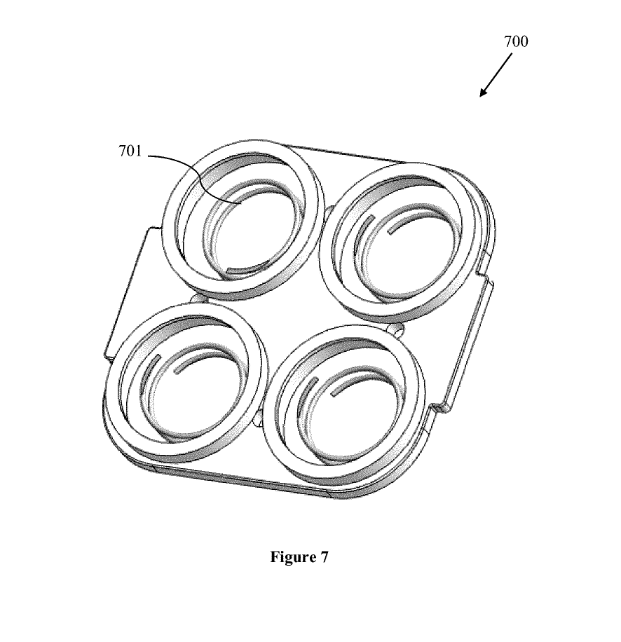

One embodiment of the invention discloses the sterile barrier assembly (303) may comprises of a housing (401) having a top surface (421) and a bottom surface (419) and at least one floating plate (423) (shown in FIG. 4 (b)) positioned on the top surface (421). The at least one floating plate (shown in FIG. 5(a)) having at least one rotatable body (shown in FIG. 5(a)). Detailed explanation of the floating plate having rotatable body is provided in description of accompanying figures. The sterile barrier assembly (303) may further comprise of a compression mechanism (shown in FIG. 7) and detailed explanation of the same is provided in the description of accompanying figures.

The sterile barrier assembly (303) may comprises of at least one locking lug (403) and at least one female guide (415). The at least one locking hugs (403) is arranged on one end of the lower surface (419) of the sterile barrier assembly (303) and the at least one female guides (415) is arranged on the opposite side to the one or more locking hugs (403) of the lower surface (419) of the sterile barrier assembly (303).

The at least one locking lug (403) may comprise of a cylindrical profile with grooves on its outer circumference. The cylindrical profile may have cut outs on its outer circumference. The at least one female guide (415) may comprise of a guiding slot and in particular a rectangular guiding slot. According to a specific embodiment, the sterile barrier assembly (303) comprises of at two locking lugs (403, 425) spaced apart from each other on one end of the lower surface (419) and two female guides (415, 427) spaced apart from each other on opposite end to the locking lugs (403, 425) on the lower surface (419).

The sterile barrier assembly (303) may be made of any suitable resilient material such as a metal or an alloy. The material for the sterile barrier assembly (303) can be selected from a group consisting aluminum, steel, iron, nickel, copper, zinc, tin, or any combination thereof. In accordance to a specific embodiment of the invention, the sterile barrier assembly (303) is made of aluminum. The sterile barrier assembly (303) may be painted or may have a protective coating such as alloy coating. In accordance with an embodiment, the process of anodizing may be used to coat the sterile barrier assembly (303) such as to form a protective coating of aluminum oxide on the surface of the sterile barrier assembly (303). The sterile barrier assembly (303) may be of any suitable size that can be conveniently attached to the actuator assembly (305) without affecting ease of the surgical operation. The sterile barrier assembly (303) may be of a suitable thickness providing sufficient strength.

The sterile barrier assembly (303) may be of any suitable shape such that the ease of affixing the sterile barrier assembly (303) is maintained. In accordance with an embodiment of the invention, the sterile barrier assembly (303) is substantially of a square shaped plate where a bottom end of the sterile barrier assembly (303) comprises a larger protrusion than a top end of the sterile barrier assembly (303). Detailed explanation of the sterile barrier assembly (303) is provided in description of accompanying figures.

The actuator assembly (305) may comprises of a housing (417), at least one male guides (429) having a protruding profile, at least one locking notches (409) defining a recess (405), one or more locking plates (407), and one or more driving elements (413). The one or more locking notches (409) are arranged on one end of an upper surface (421) of the actuator assembly (305) and the one or more male guides (429) are arranged on the opposite side (of the one or more locking notches (409) of the upper surface (421) of the actuator assembly (305). The actuator assembly (305) may include various mechanical motors and electricals connections to facilitate the motion of driving elements (413) when surgeon operates at the surgeon console (117) to command the surgical instruments during the surgery.

According to a specific embodiment, the actuator assembly (305) comprises of two male guides (411, 429) spaced apart from each other on one end of the upper surface (421) and two locking notches (409, 431) spaced apart from each other on opposite end to the male guides (411, 429) on the upper surface (421). Each of the two locking notches (409, 431) defining a recess (405, 433).

The actuator assembly (305) may be made of any suitable resilient material such as a metal or an alloy. The material for the actuator assembly (305) can be selected from a group consisting aluminum, steel, iron, nickel, copper, zinc, tin, or any combination thereof. In accordance to a specific embodiment of the invention, the actuator assembly (305) is made of aluminum. The actuator assembly (305) may be painted or may have a protective coating such as alloy coating. In accordance with an embodiment, the process of anodizing may be used to coat the actuator assembly (305) such as to form a protective coating of aluminum oxide on the surface of the actuator assembly (305). The actuator assembly (305) may be of any suitable size that can be conveniently attached to the tool interface assembly (200) without affecting ease of the surgical operation. The actuator assembly (305) may be of a suitable thickness providing sufficient strength.

The actuator assembly (305) may be of any suitable shape such that the ease of affixing the actuator assembly (305) is maintained. In accordance with an embodiment of the invention, the actuator assembly (305) is substantially of a rectangular shape where the body of the actuator assembly (305) is substantially tapered towards a top end of the actuator assembly (305). Detailed explanation of the actuator assembly (305) is provided in description of accompanying figures.

In a specific embodiment of the invention, the sterile adapter assembly (303) is releasably attached with the actuator assembly (305) by means of an interlocking between the two locking notches (409, 431) of the actuator assembly (305) and the two locking lugs (403, 425) of the sterile adapter assembly (303) and the two male guides (411, 429) of the actuator assembly (305) and the two female guides (415, 427) of the sterile adapter assembly (303). During this locking mechanism, the locking lugs (403, 425) of the sterile adapter assembly (303) is received by the respective grooves (405) of the respective locking notches (409, 431), and simultaneously the one or more male guides (411, 429) of the actuator assembly (305) fits into the guiding slot of the female guides (427, 415) of the sterile adapter assembly (303), which locks the sterile adapter assembly (303) to the actuator assembly (305). While unlocking the sterile adapter assembly (303) from the actuator assembly (305), the locking notches (409, 431) are pressed which allows the locking lugs (403, 425) of the sterile adapter assembly (303) to release thereby detaching the sterile adapter assembly (303) from the actuator assembly (305) easily. The aforesaid locking mechanism is very fast, reliable and ergonomic which is very important during the exchange of tools during the robotic assisted surgery.

FIG. 4(b) illustrates a rear view of the actuator assembly and the sterile adapter assembly in the detached position in accordance with an embodiment of the invention. FIG. 4(b) clearly illustrates the two female guides (415, 427) and the one or more driving elements (413) with relation to the sterile adapter assembly (303) and the actuator assembly (305) respectively.

FIG. 4(c) illustrates a view of the actuator assembly and the sterile adapter assembly in the locked position in accordance with an embodiment of the invention.

In an embodiment, the at least one notch (409) of the actuator assembly (305) in a first non-locking position (as illustrated in FIG. 4a) receives the at least one engaging lug (403) of the sterile adapter assembly (303) to move from the first non-locking position to a first locked position (A) and the at least one female guide (415) of the sterile adapter assembly (303) in a second non-locking position (as illustrated in FIG. 4a) receives the at least one male guide (429) of the actuator assembly (305) to move from the second non-locking position to a second locking position (B) thereby interlocking the sterile adapter assembly (303) with the actuator assembly (305).

FIG. 5(a) illustrates a top view of the sterile adapter assembly in accordance with an embodiment of the invention. The sterile adapter assembly (303) may include the housing (401) having the upper surface (421) and the lower surface (419), a memory device (507), the at least one floating plate (423), where the floating plate (423) may comprises of one or more rotatable body (505) resiliently mounted on each floating plate (423). In an embodiment, four rotatable body (505a), (505b), (505c), (505d) are mounted on one floating plate (423) and are spaced apart from each other such that the rotation of each rotatable body (505a), (505b), (505c), (505d) is not affected by the rotation of the neighboring rotatable body. According to a specific embodiment, the rotatable body (505a), (505b), (505c), (505d) have a circular profile.

In another embodiment, each of the rotatable body (505a), (505b), (505c), (505d) may comprise of a compression mechanism (700) enclosed within a housing of the rotatable body (505a), (505b), (505c), (505d). Details of the compression mechanism (700) is discussed with the description of the accompanying FIG. 7.

In another embodiment, each of the rotatable body (505a), (505b), (505c), (505d) may be made of any suitable resilient material such as a metal or an alloy. The material for the rotatable body (505a), (505b), (505c), (505d) can be selected from a group consisting aluminum, steel, iron, nickel, copper, zinc, tin, or any combination thereof. In accordance to a specific embodiment of the invention, each of the rotatable body (505a), (505b), (505c), (505d) is made of aluminum. The rotatable body (505a), (505b), (505c), (505d) may be painted or may have a protective coating such as alloy coating. In accordance with an embodiment, the process of anodizing may be used to coat the rotatable body (505a), (505b), (505c), (505d) such as to form a protective coating of aluminum oxide on the surface of the rotatable body (505a), (505b), (505c), (505d). The rotatable body (505a), (505b), (505c), (505d) may be of any suitable size that can be conveniently affixed to the floating plate (423) without affecting ease of the mechanical operation. The rotatable body (505a), (505b), (505c), (505d) may be of a suitable thickness providing sufficient strength.

In another embodiment, the at least floating plate (423) may comprise of openings into which the rotatable body (505a), (505b), (505c), (505d) can be affixed. The rotatable body (505a), (505b), (505c), (505d) may be glued onto the floating plate (423) or may be rotatably secured, bolted, riveted, screwed or a combination thereof to the floating plate (423).

The memory device (507) may be any readable memory devices such as flash memory, EEPROM, or the like. The memory device (507) is being capable of storing the various data but not limited to surgical instruments type, combability information and the like. Each rotatable body (505a), (505b), (505c), (505d) is capable of being electromechanically coupled with the surgical instrument/tool.

FIG. 5(b) illustrates a bottom view of the sterile adapter assembly in accordance with an embodiment of the invention. The bottom view of the sterile adapter assembly (303) shows the bottom surface of the floating plate (423) and the bottom surface of the rotatable body (505a), (505b), (505c), (505d) mounted on the floating plate (423). Each rotatable body (505a), (505b), (505c), (505d) is capable of being electromechanically coupled with the driving elements (413) (shown in FIG. 4 (b)).

The sterile adapter assembly (303) also comprises of two locking lugs (403, 425) on one end of the bottom surface (419) and two female guides (415, 427) on the opposite end of the bottom surface (419).

In a specific embodiment of the invention, the sterile adapter assembly (303) is releasably attached with the actuator assembly (305) by means of an interlocking between the two locking notches (409, 431) of the actuator assembly (305) and the two locking lugs (403, 425) of the sterile adapter assembly (303) and the two male guides (411, 429) of the actuator assembly (305) and the two female guides (415, 427) of the sterile adapter assembly (303).

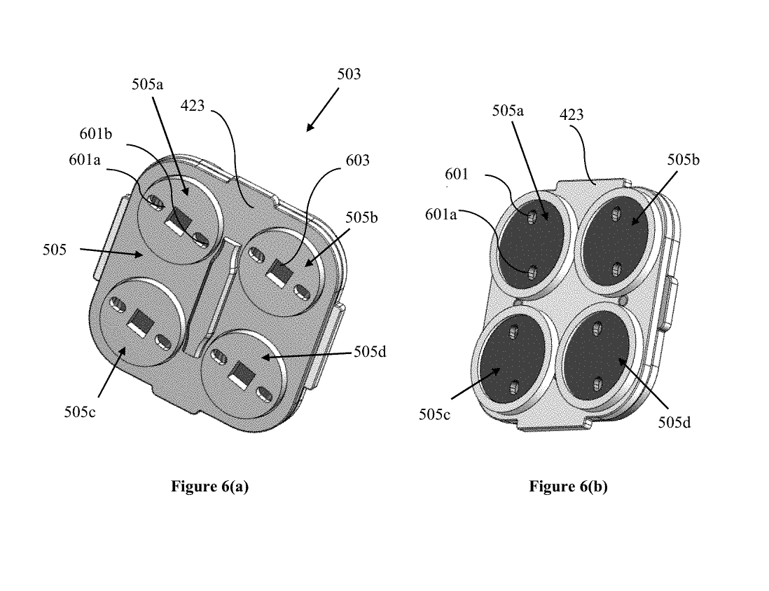

Referring to FIGS. 6 (a) and 6 (b), the upper side and bottom side of the floating plate (423) with the rotatable body (505a), (505b), (505c), (505d) is illustrated according to an embodiment of the invention. As shown in FIG. 6 (a), each rotatable body (505a), (505b), (505c), (505d) may include one or more hollow openings through its circumferential surface. In a specific embodiment, each rotatable body (505a), (505b), (505c), (505d) comprises of two hollow openings (601a), (601b). Each rotating body (505a), (505b), (505c), (505d) on the floating plate (423) may further include a recess (603) sandwiched between the openings (601a), (601b). The distance between the openings (601a), (601b) from the each other and from the recess (603) may vary depending upon the configuration of each rotatable body (505). The floating plate (423) (as shown in FIG. 6 (b)) shows the bottom view of each rotatable body (505a), (505b), (505c), (505d). Further, each of the floating plates (423) is capable of moving up and down within the sterile barrier assembly (303).

The openings (601a), (601b) are configured to receive and engage with one or more pins of the driving elements (413) of the actuator assembly (305). The one or more pins of the driving elements (413) are configured to snap-fit the openings (601a), (601b) of the rotatable body (505a), (505b), (505c), (505d). The configuration of the circumferential surface of the driving elements (413) is substantially similar to the circumference of the openings (601a), (601b) of the rotatable body (505a), (505b), (505c), (505d).

In another embodiment, the floating plate (423) is affixed within the housing (401) such that the floating plate (423) is engaged with the top surface (421) and the bottom surface (419).

FIG. 7 illustrates a floating plate without the rotating body according to an embodiment of the invention. A compression mechanism (700) is positioned in the recess (not shown) provided where the rotatable body is supposed to be positioned. According to a specific embodiment, the compression mechanism is a spring (701). The spring (701) may be integrated with the rotatable body (505a), (505b), (505c), (505d) and may facilitates the rotatable body (505a), (505b), (505c), (505d) to compress and expand along its axis.

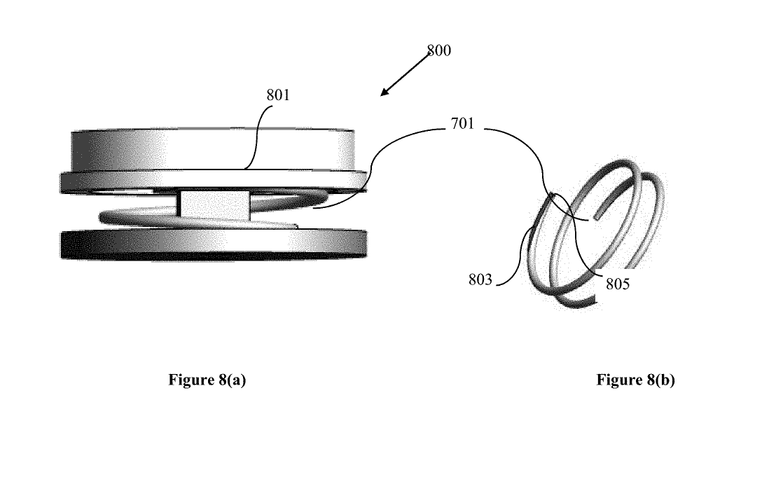

Now referring to FIGS. 8 (a) and 8 (b), a single rotatable body (800) is illustrated according to an embodiment of the invention. The rotatable body (800) comprises of an upper body (801) and a lower body (803) operationally connected by a guiding bar (805). Further, the spring (701) is sandwiched between the upper body (801) and lower body (801) and coiled around the guiding bar (805). The spring (701) and guide bar (805) mechanism provide additional flexibility between the upper body (801) and lower body (803) of the rotatable body (800). Such arrangement provides efficient transfer to commands from the surgeon console to the actuator assembly (305) to control the movement of the surgical instruments.

In a specific embodiment, when the sterile adapter assembly (303) is engaged with the actuator assembly (305), the spring (701) facilitates in locking of the sterile adapter assembly (303) with the actuator assembly (305). More specifically, when the sterile adapter assembly (303) is engaged with the actuator assembly (305), one or more pins of the driving elements (413) and the openings (601a), (601b) of the rotating body (505) are misaligned and more often, they are not engaged with each other. In such situation, the spring (701) between the rotating body (505) allows the sterile adapter assembly (303) to get locked with the actuator assembly (305) without actually engagement of the one or more pins of the driving elements (413) with the openings (601a), (601b) of the rotating body (505). After, locking of the sterile adapter assembly (303) with the actuator assembly (305), homing is done (with the help of the tension of spring) in which the one or more pins of the driving elements (413) are actually engaged with the openings (601a), (601b) of the rotating body (505).

In another embodiment, the sterile adapter assembly (303) is integrated with a sterile drape (not shown) for draping portions of a robotic surgical system (particularly the robotic arms (103a), (103b), (103c), (103d)) to maintain a sterile barrier between a sterile surgical field and a non-sterile robotic system while also providing an interface for transferring mechanical and electrical energy and signals between a surgical instrument and the robotic surgical system. In one embodiment, sterile adapter assembly (303) may be permanently attached to the sterile drape by means of a film adhesive material which is impulse heat sealed and/or attached using adhesive film to the sterile drape.

In another embodiment, the sterile adapter assembly (303) further includes a pair of supports (not shown) that serve to properly align, position, and retain a surgical instrument on the upper side of the sterile adapter assembly (303) for engagement with an instrument manipulator. The rotatable body (505a), (505b), (505c), (505d) helps the instrument manipulator to align the surgical instrument on the sterile adapter assembly (303).

The foregoing descriptions of exemplary embodiments of the present invention have been presented for purposes of illustration and description. They are not intended to be exhaustive or to limit the invention to the precise forms disclosed, and obviously many modifications and variations are possible in light of the above teaching. The exemplary embodiment was chosen and described in order to best explain the principles of the invention and its practical application, to thereby enable others skilled in the art to best utilize the invention and various embodiments with various modifications as are suited to the particular use contemplated. It is understood that various omissions, substitutions of equivalents are contemplated as circumstance may suggest or render expedient, but is intended to cover the application or implementation without departing from the spirit or scope of the claims of the present invention.

Benefits, other advantages, and solutions to problems have been described above with regard to specific embodiments. However, the benefits, advantages, solutions to problems, and any component(s) that may cause any benefit, advantage, or solution to occur or become more pronounced are not to be construed as a critical, required, or essential feature or component of any or all the claims.

While specific language has been used to describe the disclosure, any limitations arising on account of the same are not intended. As would be apparent to a person in the art, various working modifications may be made to the apparatus in order to implement the inventive concept as taught herein.

1. A sterile adapter assembly comprising:
a housing;
a floating plate positioned within the housing;
at least one rotatable body having a circumferential surface affixed on the floating plate, the at least one rotatable body having at least one opening capable of receiving at least one pin of a driving element of an actuator assembly; and
a compression mechanism positioned inside the at least one rotatable body to align the at least one pin of the driving element of the actuator assembly with the at least one opening of the at least one rotatable body.

2. The sterile adapter assembly as claimed in claim 1, wherein the housing having a top surface and a bottom surface and the floating plate is affixed within the housing such that the floating plate is engaged with the top surface and the bottom surface.

3. The sterile adapter assembly as claimed in claim 1, further comprising at least one engaging lug positioned on the bottom surface for engaging when in operation with at least one notch of an actuator assembly.

4. The sterile adapter assembly as claimed in claim 1, further comprising at least one female guide positioned opposite to the at least one engaging lug on the bottom surface, the at least one female guide engages when in operation with at least one male guide of the actuator assembly.

5. The sterile adapter assembly as claimed in claim 1, further comprising a memory device capable of recording data.

6. The sterile adapter assembly as claimed in claim 1, further comprising at least two rotatable bodies having a circumferential surface affixed on the floating plate wherein the at least two rotatable bodies are spaced apart from each other such that the rotation of each of the at least two rotatable bodies is not affected by the rotation of neighboring rotatable body.

7. The sterile adapter assembly as claimed in claim 1, wherein the compression mechanism is a spring.

8. The sterile adapter assembly as claimed in claim 1, wherein the sterile adapter assembly is integrated with a sterile drape for draping portions of a robotic surgical system.

9. The sterile adapter assembly as claimed in claim 8, wherein the sterile adapter assembly is permanently attached to the sterile drape by means of a film adhesive material.

10. The sterile adapter assembly as claimed in claim 1, wherein the sterile adapter assembly further includes a pair of supports that serve to properly align, position, and retain a surgical instrument on the upper side of the sterile adapter assembly for engagement with an instrument manipulator.
